(19) United States
(12) Patent Application Publication
(10) Pub. No.: US 2011/0226652 A1
(43) Pub. Date: Sep. 22, 2011

(54) Packing for Inserter

(76) Inventors: Richard Morgan Hickmott, Helsingor (DK); Henrik Jeppesen, Holte (DK)

(21) Appl. No.: 13/121,290

(22) PCT Filed: Sep. 28, 2009
(86) PCT No.: PCT/EP09/62500

§ 371 Date: Jun. 7, 2011

Related U.S. Application Data
(60) Provisional application No. 61/100,860, filed on Sep. 29, 2008.

(51) Int. Cl. A61B 19/02

(52) U.S. Cl. 206/438

(57) ABSTRACT

The invention concerns a kit comprising a medical device having an insertion needle and a packing providing a sterile protection for the medical device, such a medical device can be used for an infusion device providing continued medication with e.g. insulin to a patient. More precisely the invention relates to an assembly comprising a medical device comprising a mounting pad having an adhesive surface and an inserter which inserter comprises an insertion needle and a packing providing a sterile protection for the medical device which packing partly comprises a rigid material (1, 2, 7, 8, 5) and partly a soft material (10), wherein the rigid material is constructed of two angularly positioned and rigidly joined surfaces, a first and a second surface (1, 2), which surfaces (1, 2) cover two sides of the medical device (3, 4) which sides face two different directions and the non-adhesive surface of the mounting pad is not covered by a rigid surface.

### TECHNICAL FIELD OF THE INVENTION

The invention concerns a kit comprising a medical device having an insertion needle and a packing providing a sterile protection for the medical device.

### BACKGROUND OF THE INVENTION

Single use packages for medical devices having an insertion needle are well known. The single-use packages should preferably be inexpensive to manufacture and they should be able to provide a sterile environment for the medical device until the device is unpacked by the user and the insertion needle without further sterilization of the medical device can safely penetrate the patient's skin.

WO 2007/122207 shows a packaging for a skin-mountable device comprising a coated seal member. The assembly comprises a skin-mountable device (10) with an adhesive mounting surface (31), the device being arranged in a packaging (20) comprising an opening with a surrounding portion and being closed with a seal member (40) releasably attached to the surrounding portion (22), thereby providing a closed space for the device. The seal member has an inner surface releasably attached to the adhesive, the seal member being penetrable by a sterilizing gas, wherein the inner surface I partially coated with a material (42, 44) allowing the seal member to be peeled from the adhesive, yet allows the sterilizing gas to penetrate the seal member. In this way a seal member is provided which to a high degree has two desirable properties: being gas penetrable yet allowing the seal member to be peeled from the adhesive surface.

### SUMMARY OF THE INVENTION

The present invention provides an assembly comprising
a medical device comprising a mounting pad having an adhesive surface and an inserter which inserter comprises an insertion needle and
a packing providing a sterile protection for the medical device which packing partly comprises a rigid material and partly a soft material. The rigid material is constructed of two angularly positioned and rigidly joined surfaces, a first and a second surface which surfaces cover two sides of the medical device which sides face two different directions and the non-adhesive surface of the mounting pad is not covered by a rigid surface. That the surfaces are “rigidly joined” means that they are joined like a hinge i.e. displacement of one surface will influence on the other surface but the flexibility between the two surfaces might be regulated by the choice of material and thickness of material. The adhesive surface and the non-adhesive surface of the mounting pad are positioned opposite each other, this means that when the non-adhesive surface of the mounting pad is not covered by the rigid surface, the non-adhesive surface face a potential opening through which the medical device can be removed from the packing.

According to one embodiment the second surface provides a contact surface for the adhesive surface of the mounting pad. That the rigid material provides a contact surface means that the rigid material either touches the adhesive surface directly providing a protective surface which should be removed before use or that the rigid material supports a protective layer which protective layer is in direct contact with the adhesive surface. The rigid material normally covers the whole area of the adhesive surface of the mounting pad but it might only be in actual contact with the adhesive surface or its protective layer in some designated areas or points.

According to one embodiment the packing is provided with side parts made of rigid material which side parts connect the two rigidly joined surfaces thereby forming a non-flexible structure. This embodiment makes it easier for the individual packing to withstand outside forces impacting the packing. Also it is simpler to place a soft material which provides sterile conditions inside the packing.

According to one embodiment the packing is internally provided with a distance piece inserted between the medical device and the first surface or a side part of the packing. The distance piece makes it possible to get hold of the medical device when the medical device is placed in the packing before use.

According to one embodiment the edge of the rigid material has a flat circumferential portion (5) surrounding an opening defined by the edge of the rigid material to which circumferential portion (5) a protective seal of soft material (10) can be releasably attached. The medical device can be removed from the packing and replaced in the packing through the opening formed by the edge of the rigid material.

According to one embodiment the rigid material is moulded in one piece.

According to one embodiment the packing comprises means for reclosing. The packing need not necessarily be completely closed but it should be closed to a degree where the medical device is secured in the packing after use. According to this embodiment the means for reclosing can comprise adhesive and/or mechanical means.

If the means for reclosing are adhesive they can be provided by an adhesive or sticky material placed between the soft material and the circumferential portion. The sticky material might be placed all along the circumferential portion 5 or on the soft material during manufacturing or it can be placed in portions. The sticky material can e.g. be PA (polyamide).

If the means for reclosing are mechanical they can be provided by one or more openings in the soft material which openings correspond to protruding parts of the rigid material.

According to one embodiment the inserter of the medical device is releasably attached to a base part which base part is to be positioned on a patient's skin. The base part is of such a size that a delivery part comprising a reservoir can be attached directly to the base part. A reservoir normally contains 1-10 ml.

According to another aspect of the invention, the invention comprises a packing providing a sterile protection for a medical device stored in the packing which packing partly comprises a rigid material and partly a soft material. The rigid material is constructed of two angularly positioned and rigidly joined surfaces, a first and a second surface which surfaces cover two sides of the medical device which sides face two different directions and the non-adhesive surface of the mounting pad is not covered by a rigid surface.

### DEFINITIONS

When relative terms as “upper”, “lower”, “right”, “left”, “horizontal”, “vertical or similar terms are used, the terms refer to the appended figures and not necessarily to a situation of actual use.

Rigid material—cannot be penetrated by an insertion needle and is able to sustain a given shape.

Soft material—might be penetrated by an insertion needle, can e.g. be penetrated by sterilizing gas, can be folded or bend by the user i.e. the material will not necessarily sustain a given shape during use.

### BRIEF DESCRIPTION OF THE DRAWINGS

A detailed description of embodiments of the current invention will be made with reference to the accompanying figures, wherein like numerals designate corresponding parts in different figures.

FIG. 1 shows a first embodiment of the rigid part of a packing in which a medical device comprising an inserter combined with a mounting part is placed.

FIG. 2 shows a second embodiment of the rigid part of a packing in which a medical device comprising an inserter combined with a mounting part is placed.

FIG. 3 shows the first embodiment of the rigid part of a packing without the medical device.

FIG. 4 shows the second embodiment of the rigid part of a packing without the medical device.

FIG. 5 shows how the first or second embodiment of the packing can be nested if ten units are packed together.

FIGS. 6A and 6B show the first embodiment of the packing seen respectively from below and provided with a soft protection layer.

FIGS. 7A and 7B show the second embodiment of the packing seen respectively from below and provided with a soft protection layer.

FIGS. 8A and 8B shows how soft and rigid materials can be combined and an embodiment of a mechanical reclosing of a lid on the packing.

FIGS. 9A and 9B illustrates a medical device in the form of an inserter attached to a base plate through which a cannula part can be inserted.

### DETAILED DESCRIPTION OF THE INVENTION

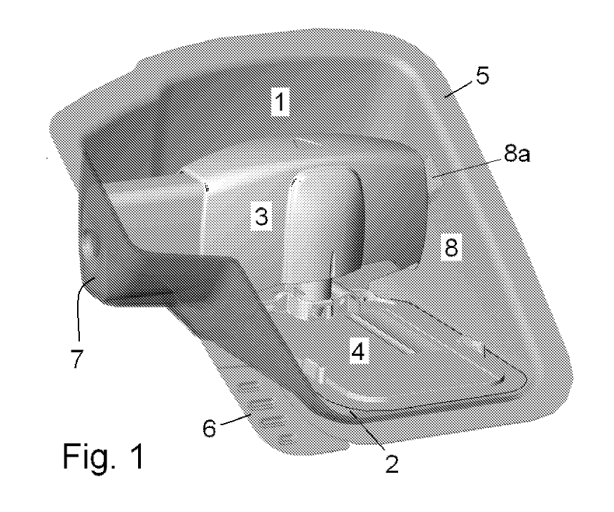

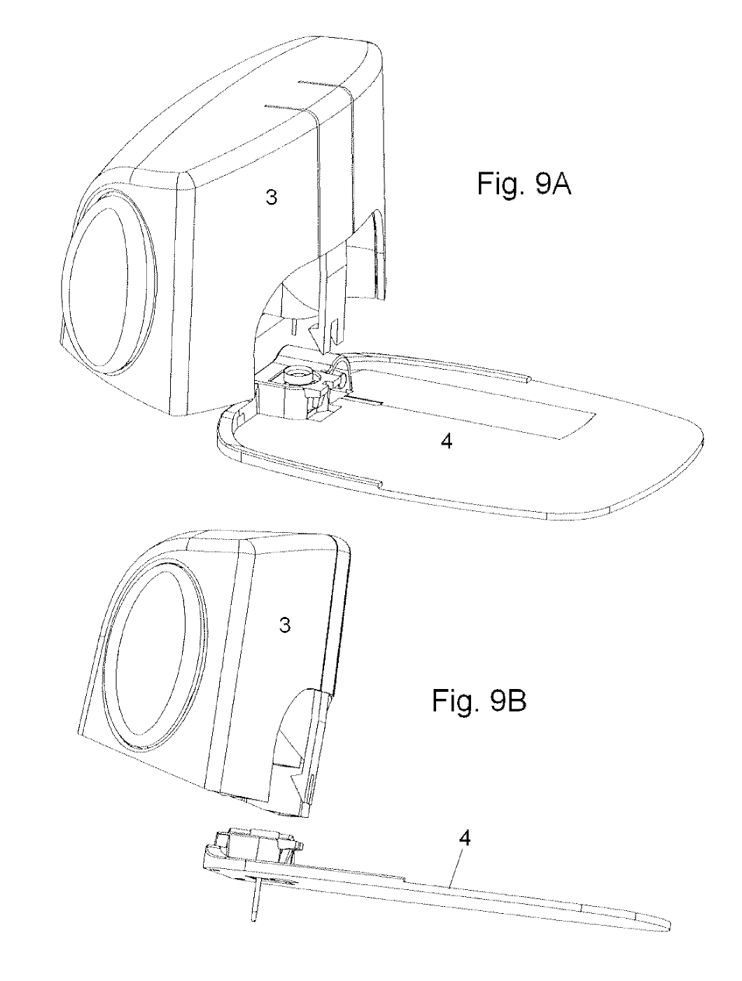

FIG. 1 shows a first embodiment of an assembly according to the invention which assembly comprises an inserter 3 combined with a base part 4 provided with a mounting pad and a packing. The inserter which comprises an insertion needle and is combined with a base part which is to be attached to a patient is illustrated in more detail in FIGS. 9A and 9B. Only the rigid material of the packing is shown in FIG. 1 as a cover of soft material has been removed in order to be able to see the inserter inside the packing.

The rigid material is constructed of two angularly positioned and rigidly joined surfaces, a first and a second surface 1, 2. The packing is further provided with two connecting side parts 7, 8 and the edge of the four sides of rigid material has a flat circumferential portion 5 surrounding an opening defined by the edge of the rigid material. A protective seal of soft material 10 can be releasably attached to the circumferential portion 5. A part 6 of the circumferential portion 5 can be detached from the rigid material and will then function as a handle which handle can be unreleasably attached to the protective seal of soft material 10. In FIG. 1 the handle 6 is still in the position which it holds before use while the protective seal of soft material has been removed, this is not a situation which will normally occur during use but the packing is shown without the protective seal in order to illustrate the inside of the packing. The packing is internally provided with a distance piece 9 inserted between the inserter 3 and the first surface 1 of the packing. The distance piece 9 makes it possible for the user to grab hold of the inserter 3 with a tweezers-like grip of e.g. the index finger and the thumb. As an alternative to a distance piece 9 the rigid material of the packing could be provided with a finger grip space i.e. a local widening of the cross-section of the packing along the first surface which provides enough room to make it possible for the user to put one or two fingers behind the medical device. In FIG. 1 the second surface 2 functions as a support on which support the inserter including the base part can be left while the user e.g. arranges closing or sterilizes the injection site. The surfaces 1, 2 cover two sides of the inserter 3/base part 4 device, the upright side 1 covers one side of the inserter 3 and the support side 2 covers the mounting side of the base part 4. According to this embodiment the non-adhesive side of the base part 4 which is pointing upwards in FIG. 1 is not covered by a rigid surface but will be covered by a soft material which has to be removed before it is possible to remove the inserter incl. the base part from the packing. The non-adhesive side of the mounting pad is the side opposite the adhesive surface i.e. if the adhesive surface is facing downwards the non-adhesive side is facing upwards.

That the packing has two sides 1, 2 made of a rigid material makes it easy for the user to grab hold of the packing with one hand and at the same time remove the medical device with the other hand and it makes it possible for the user to place the packing on a non-sterile surface and e.g. push down on the packing while a cover made of a soft material is torn off. At the same time it is easy to remove a medical device having an irregular shape from the packing.

The second surface can provide a contact surface for the adhesive side of the mounting pad of the base part 4 i.e. the mounting pad adheres to the second surface 2 while the base part 4 and the attached mounting pad is placed in the packing and the second surface 2 provides a protecting layer for the adhesive surface. Alternatively the mounting pad can be provided with a separate release layer which provides a protecting layer for the adhesive surface and which release layer is removed together with the base part 4 when the inserter 3 including the base part 4 is removed from the packing.

The packing of the assembly shown in FIG. 1 is provided with connecting side parts 7, 8 made of rigid material. The rigid material used to connect the rigidly joined first and second surface 1, 2 is according to this embodiment the same material as the first and second surface 1, 2 are made of, actually all four sides 1, 2, 7, 8 can be moulded as one piece out of the same material. The connecting side parts 7, 8 connect the two rigidly joined surfaces 1, 2 and thereby form a packing having a non-flexible structure. This embodiment makes it easier for the individual packing to withstand outside forces impacting the packing. Also it is simpler to place a soft material which provides sterile conditions inside the packing along the edge as the flat edge 5 is in a plane surface.

In the embodiment of FIG. 1 the first connecting side part 7 is formed with a protruding hollow part which is able to surround the activation handle of the inserter 3 and thereby fit closely around the inserter 3 and the base part 4. The second connecting side part 8 is formed as a plane surface having the form of a flat, right angled triangle attached respectively to the first surface 1 and the second surface 2 along each of the right angled sides. On the internal surface the second connecting side part 8 is provided with a small protruding part 8a which help keeping the inserter in position while the inserter 3 is in the packing as the protruding part 8a prevents the inserter 3 and the attached base part 4 to move towards the front edge of the packing.

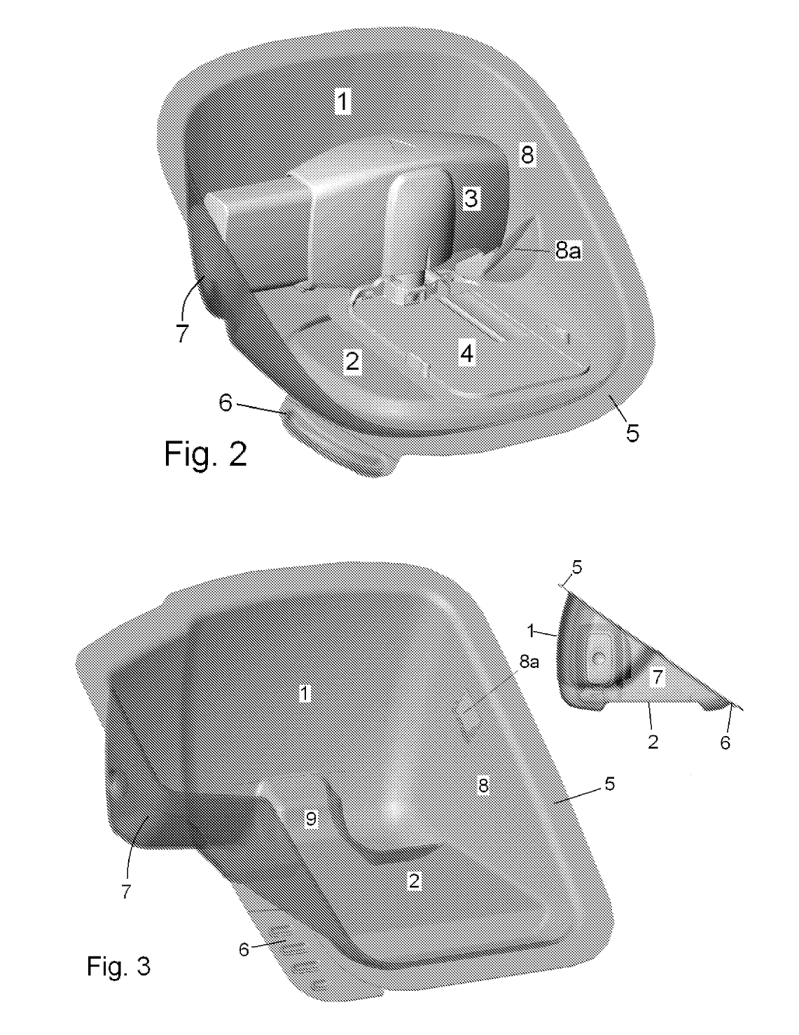

FIG. 2 shows a second embodiment of an assembly according to the invention which assembly also comprises a packing and an inserter 3 combined with a base part 4 provided with a mounting pad. As in FIG. 1 only the rigid material of the packing is shown in FIG. 2.

As for the first embodiment the rigid material is constructed of two angularly positioned and rigidly joined surfaces, the first and a second surface 1, 2 and the packing is also provided with two connecting side parts 7, 8 and the edge of the four sides of rigid material has a flat circumferential portion 5 for releasable attachment of a protective seal of soft material 10. Like in the first embodiment a part 6 of the circumferential portion 5 can be detached from the rigid material and can function as a handle which handle can be unreleasably attached to the protective seal of soft material 10. In FIG. 2 the handle 6 is also still in the position which it holds before use while the protective seal of soft material has been removed. The packing is internally, i.e. on the internal surface, provided with two distance pieces 9 placed respectively on the first and the second connecting side parts 7, 8, two small protruding parts 8a are placed opposite each distance piece 9 and the oppositely place parts holds the inserter in a well-defined position and makes it possible for the user to grab hold of the inserter 3 with a tweezers-like grip of e.g. the index finger and the thumb.

The second surface 2 also functions as a support for the medical device according to the second embodiment i.e. the medical device can be left on this support while the user e.g. arranges closing or sterilizes the injection site.

The packing of the assembly shown in FIG. 2 is also provided with side parts 7, 8 made of rigid material. The rigid material used to connect the rigidly joined first and second surface 1, 2 is according to this embodiment made of the same material as the first and second surface 1, 2 are made of, actually all four sides 1, 2, 7, 8 are moulded as one piece out of the same material. The connecting side parts 7, 8 connect the two rigidly joined surfaces 1, 2 and thereby form a packing having a non-flexible structure.

In the embodiment of FIG. 2 the two connecting side parts 7, 8 are both formed as a plane surface having the form of a flat, right angled triangle attached respectively to the first surface 1 and the second surface 2 along each of the right angled sides.

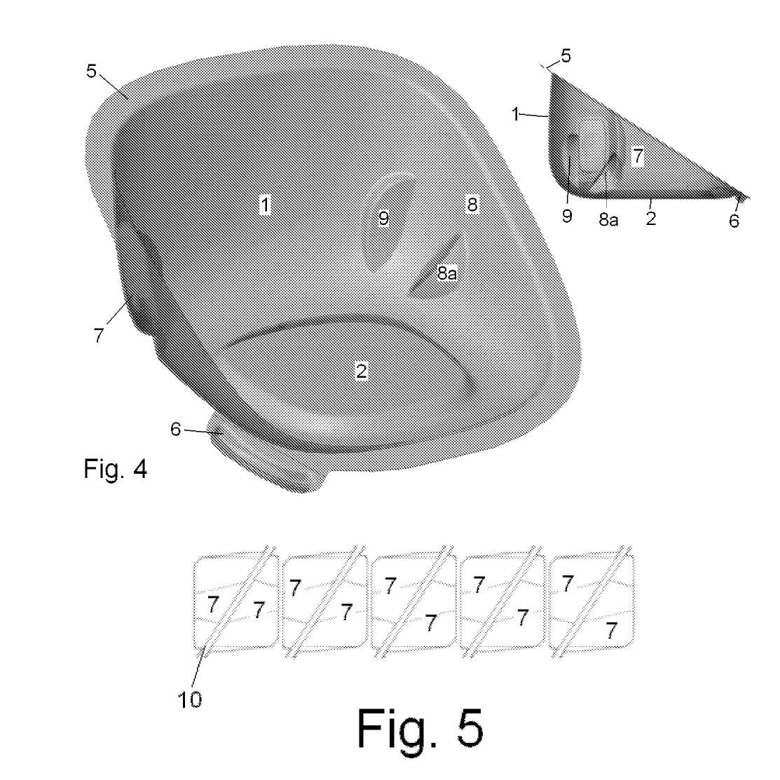

FIGS. 3 and 4 show the rigid material of the packing of respectively the first and the second embodiment without the medical device placed in each of the packing. This view makes it possible to see the distance pieces 9 in each embodiment.

FIGS. 3 and 4 also show each embodiment in a side view which illustrates how the construction comprising the two hinged surfaces 1, 2 connected via the connecting side parts 7, 8 provides the product with a triangular profile when seen from the side.

FIG. 5 illustrates how both the first and the second embodiments can be packed into a packet of ten. A multi-packet containing an equal number of units i.e. 2, 4, 6, 8, 10 etc. will be very resistant to impacts from the surroundings and will take up very little space during storage i.e. the packing is easy to pack in large amounts and provides minimal material usage.

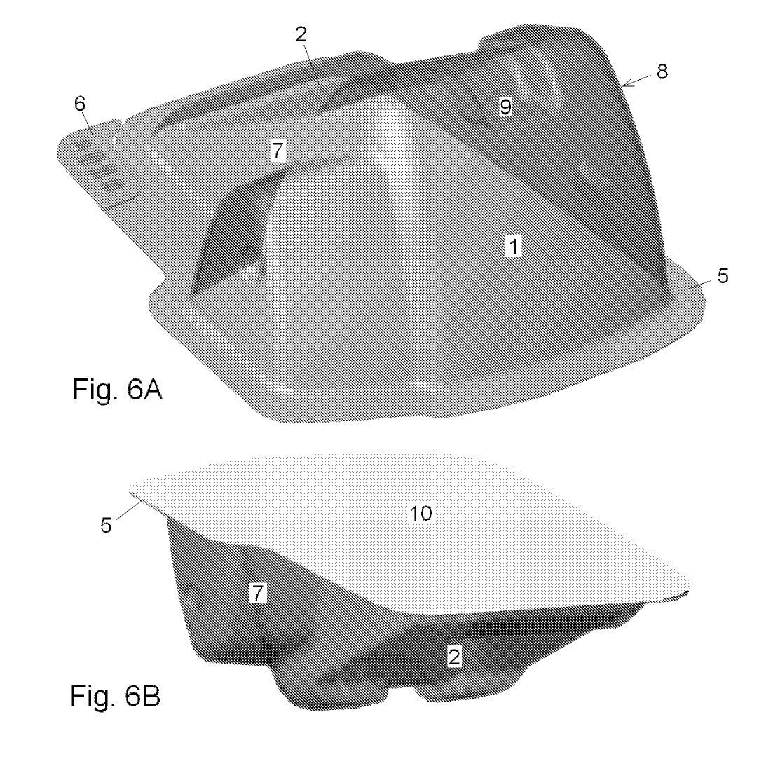

FIGS. 6A and 6B show respectively a top view and a bottom view of the first embodiment of the assembly according to the invention. In these views the rigid and the soft parts of the packing is joined and from FIG. 6B it can be seen how the protective soft material 10 is attached to the circumferential portion 5 of the rigid material 1, 2, 7, 8.

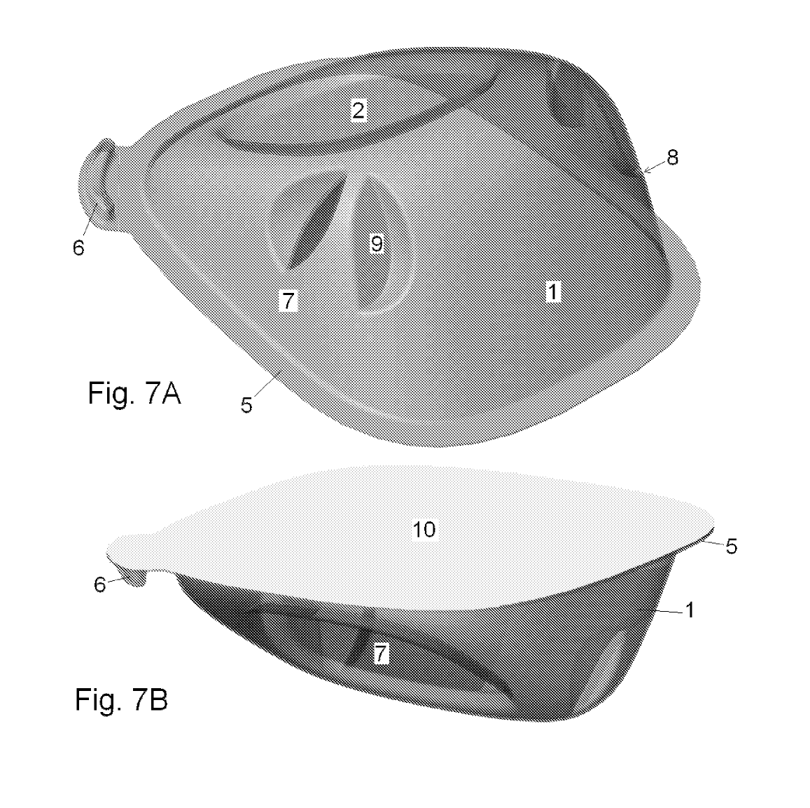

FIGS. 7A and 7B show respectively a top view and a bottom view of the second embodiment of the assembly according to the invention. In these views the rigid and the soft parts of the packing is joined and from FIG. 7B it can be seen how the protective soft material 10 is attached to the circumferential portion 5 of the rigid material 1, 2, 7, 8.

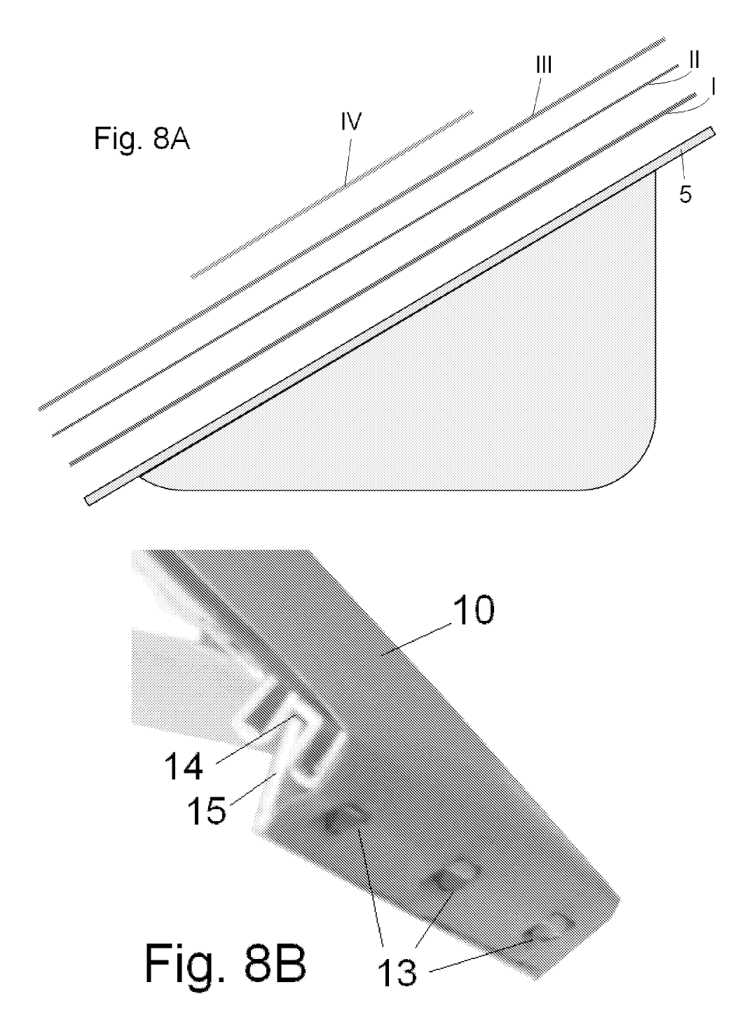

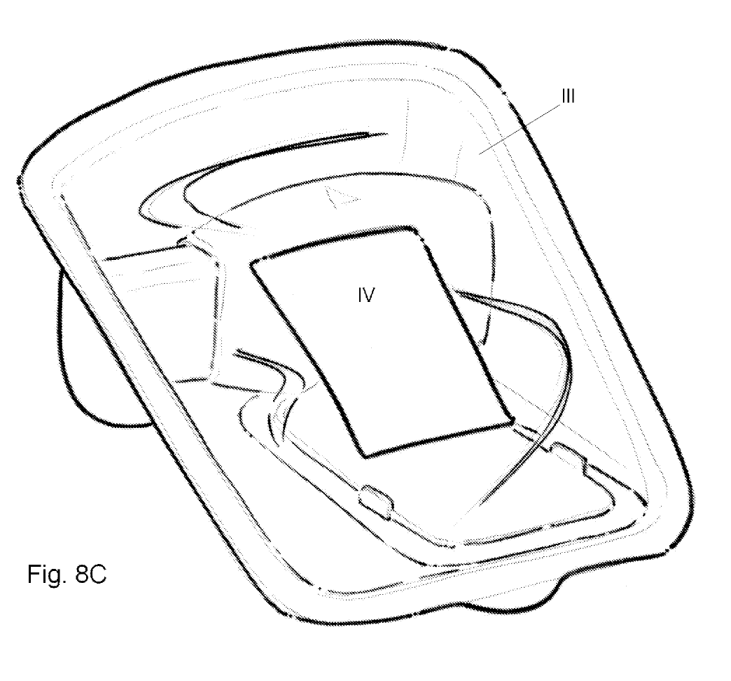

FIG. 8A illustrates the different layers which will normally be present along the circumferential portion 5 and covering the opening of the rigid material:

I. The first layer is found closest to the rigid material and consists of a melt layer e.g. made of polyethylene. This melt layer is positioned along the circumferential portion 5 of the rigid material.

II. The second layer is positioned on top of the melt layer and consists of a sticky layer e.g. made of polyamide. The melt layer is during manufacture of the packing used to weld a top layer on.

III. The third layer provides at least partly an outer surface of the packing i.e. the layer is a top layer and consists of either paper or plastic (e.g. PET) of a relatively resistant material. The top layer covers both the circumferential portion 5 and the opening of the rigid material. When the user tears of the top layer the connection to the melt layer is broken and the exposed sections of sticky layer provide portions to which the top layer can stick so the packing can be reclosed.

IV. The fourth layer is e.g. made of paper and can provide a surface which is suitable for showing e.g. the name of the manufacturer.

FIG. 8B shows a mechanism for reclosing of the open packing after use. When on the move the user may not always have access to a bin. This means that the user is required to carry the unhygienic used parts around in a bag or a pocket and this creates a need to be able to reuse the packing and preferably to reclose the packing in order to keep especially the insertion needle away from contact with other persons.

A reclosing mechanism can either be of a chemical or a mechanical type. If the reclosing is chemical the reclosing mechanism normally comprises a sticky layer II placed at least on portions of either the rigid material and/or the protective soft material 10 in positions where the protective soft material 10 can be rejoined to the rigid material after the medical device has been used and afterwards replaced inside the packing.

If the reclosing is mechanical it might be of the type shown in FIG. 8B. The lid or the top layer comprising the soft protective material 10 covers the opening provided by the circumferential portion 5. The top layer has a series of holes 13 along an edge (three holes are shown in FIG. 8B).

A part of the circumferential portion 5 of the rigid material has a trail 14 in which a folded border 15 of the top layer 10 is placed when the mechanism is closed. When the mechanism is closed protruding parts of the rigid material protrude through the holes 13 made in the top layer.

FIG. 8C shows a three-dimensional view of a packing provided with a top layer III and a fourth layer IV. The top layer corresponds to the protective soft material 10 which can be made of paper or plastic or another material with similar characteristics.

FIGS. 9A and 9B show a medical device which appropriately can be used with an assembly according to the invention. The medical device comprises an inserter 3 and a base part 4. Before the medical device is removed from the packing and put to use, the inserter 3 should be releasably attached to the base part 4, this eliminates the act where the user has to join the inserter to the base part or to a cannula part. When the inserter 3 is attached to the base part 4, which is the situation before use, the combined unit is relatively large and relatively fragile as it will also be possible to separate the inserter 3 from the base part 4 before the base part 4 has been correctly positioned on the patients' skin and the cannula part has been inserted. The packing according to the invention prevents this separation from happening during storage and transport.

The device in FIGS. 9A and 9B is shown in a situation just after insertion of the cannula part, where the inserter has come loose from the base part 4. The base part 4 comprises a plate in form of a flat mounting surface on which a reservoir and delivery means in the form of a pump and e.g. controlling means can be placed and thereby carried around by the user.

An inserter 3 and a base part 4 is known from Danish patent application PA200800185 (filing date: 8 Feb. 2008) and the embodiments of the inserter and base part described in FIGS. 12-19 of this application is incorporated herein by reference.

1. An assembly comprising:
a medical device comprising a mounting pad having an adhesive surface and an inserter, the inserter comprising an insertion needle; and
a packing providing a sterile protection for the medical device the packing comprising a rigid material portion and a soft material portion, the rigid material portion includes four sides of rigid material comprising:
two sides having angularly positioned and rigidly joined surfaces, a first and a second surface, the first and second surfaces cover two sides of the medical device respectively, the adhesive surface of the mounting pad and one side of the inserter, the two sides of the medical device face two different directions and
a non-adhesive surface of the mounting pad is not covered by a rigid surface and
two other sides connecting the rigidly joined first and second surfaces and covering opposite ends of the inserter thereby forming a non-flexible structure.

2. An assembly according to claim 1, wherein an edge of the four sides of rigid material has a flat circumferential portion for releasable attachment of the protective seal of soft material.

3. An assembly according to claim 1, wherein the second surface provides a contact surface for the adhesive surface of the mounting pad.

4. An assembly according to claim 1, wherein the first side and the second side are placed at a right angle relative to each other.

5. An assembly according to claim 1, wherein an edge of the rigid material has a flat circumferential portion surrounding an opening defined by the edge of the rigid material a protective seal of soft material can be releasably attached to the circumferential portion.

6. An assembly according to claim 1, wherein the rigid material is moulded in one piece.

7. An assembly according to claim 1, wherein the packing comprises means for reclosing.

8. An assembly according to claim 7, wherein the means for reclosing comprise adhesive or mechanical means.

9. An assembly according to claim 8, wherein the means for reclosing is provided by an adhesive or sticky material placed between the soft material and the circumferential portion.

10. An assembly according to claim 8, wherein the means for reclosing is provided by one or more openings in the soft material, the one or more openings correspond to protruding parts of the rigid material.

11. An assembly according to claim 1, wherein the inserter of the medical device is releasably attached to a base part, the base part is to be positioned on a patient's skin and the base part is sized so that a delivery part comprising a reservoir can be attached directly to the base part.

12. A packing providing a sterile protection for a medical device stored in the packing, the packing comprising a rigid material portion and a soft material portion;
the rigid material including four sides of rigid material comprising:
two angularly positioned and rigidly joined first and a second surfaces, the first and second surfaces cover two sides of the medical device respectively, an adhesive surface of a mounting pad of the medical device and one side of an inserter for the medical device the two sides face two different directions and a non-adhesive surface of the mounting pad is not covered by a rigid surface and
two side parts connecting the two rigidly joined surfaces thereby forming a non-flexible structure.
